(19) United States
(12) Patent Application Publication
(10) Pub. No.: US 2004/0170652 A1
(43) Pub. Date: Sep. 2, 2004

(54) Saponin inactivated mycoplasma vaccine

(76) Inventor: Robin Ashley John NIiholas, Addlestone (GB)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/476,508

(22) PCT Filed: May 3, 2002
(86) PCT No.: PCT/GB02/02062

(30) Foreign Application Priority Data
May 3, 2001 (GB) 0110851.3

(51) Int. Cl. C12N 1/20; A61K 39/02

(52) U.S. Cl. 424/264.1; 435/252.1

(57) ABSTRACT

A strain of Mycoplasma which is a causative agent of bovine pneumonias, which has been inactivated by treatment with an agent, which disrupts cel membranes such as saponin, is described. Preferably, the strain is Mycoplasma bovis or Mycoplasma mycoides subsp. mycoides SC. These strains can be used as vaccines. Veterinary compositions, methods of preparing these and their use in prophylaxis is also described and claimed.

The present invention relates to an inactivated strain of Mycoplasma, which is a causative agent of bovine pneumonias, which is useful as a vaccine in cattle, to methods of producing it and to its use.

The present invention particularly relates to an inactivated strain of Mycoplasma bovis or an inactivated strain of Mycoplasma mycoides subsp. mycoides SC.

Mycoplasmas are microorganisms deprived of cell walls and are, therefore, a) pleomorphic and b) resistant to antibiotics of the beta-lactamine group, such as penicillin. Mycoplasmas vary considerably in their composition of their cell membrane and in particular in the quantity of cholesterol incorporated therein. The membrane of M. mycoides SC is surrounded by a carbohydrate layer (galactan). In addition, Mycoplasmas have inherently high mutation rates and so may rapidly develop systems capable of combating inactivation (e.g. production of enzymes etc) as has been seen in their response to antibiotics where resistance has developed rapidly.

Mycoplasma bovis is a primary cause of calf pneumonia, arthritis, mastitis, eye disease and other conditions worldwide and has been estimated to cost the cattle industry millions of pounds in mortality and setback losses annually. Calf pneumonia is a complex syndrome involving bacteria, viruses and mycoplasmas. In Europe it is believed that between 25-33% of calf pneumonias involve Mycoplasma bovis.

Recent strains of Mycoplasma bovis are showing increased resistance to a range of antibiotics (Ayling R et al (2000) Veterinary record 146, 745-747) and there is, therefore, a need for an effective vaccine.

Mycoplasma mycoides subsp. mycoides SC variant is the cause of contagious bovine pleuropneumonia (CBPP). CBPP is widespread in Africa and it is also present in other regions of the world, including southern Europe, the Middle East and parts of Asia In Africa, its economic importance is particularly high in Sahelian and Sahelo-Sudanese countries. The T₁₄₄ vaccine strain, which is used extensively throughout Africa today, contains a crude, egg-passaged live strain of Mycoplasma mycoides subsp SC. It has residual virulence and causes adverse reactions in cattle. Moreover, the protection it confers is short-lasting and sometimes very poor as was seen during the Botswanan outbreaks of 1995.

Vaccines derived from inactivated strains of viruses or bacteria are well known in the art. The pathogenic microorganism is treated with a chemical, such as formaldehyde, which damages the organism to such an extent that it cannot cause infective disease in the animal to which it is administered. For example, the ability of the organism to replicate may be irreparably damaged. However, key antigenic regions of the organisms are retained, which stimulate an immune response in the animal. This response may provide a protective effect if the animal is later challenged with a potent strain of the organism.

Different inactivating agents have different modes of action in order to achieve the desired effect. Some agents bind DNA, others destroy or inactivate essential proteins and a further class of inactivating agents may disrupt the cell membrane. The efficacy of the agents in any particular case will depend upon the nature of the microorganism. Thus, the results achieved will be variable, as a result of the inherent variability of the microorganisms.

It is not obvious that the use of a particular inactivating agent will produce the desired result in any particular instance. This is especially the case in different species where the response to a particular inactivating agent can be quite different. This is particularly true in the case of complex organisms such as Mycoplasma bovis and Mycoplasma mycoides subsp. mycoides SC.

An experimental quadrivalent vaccine incorporating formalin killed antigens of Mycoplasma bovis and M. dispar together with viral and bacterial components was trialled (Howard et al (1987) Veterinary Record 121, 372-376. The vaccine included Quil A as adjuvant, and although effective, it was not successful enough to be produced commercially. Formalin inactivated vaccines incorporating M. bovis and Mannheima haemolytica (Urbaneck et al (2000) Der Praktische Tierarzt 81, 756-763) and one incorporating two strains of M. bovis (Rosenbusch R (1998) Proceedings of 12^{th} Congress of the International Organisation of Mycoplasmology, Sydney p185) have also been reported with mixed success. A formalised M. bovis vaccine against calf arthritis incorporating saponin as an adjuvant was immunogenic and reduced spread of infection to other joints in a small number of calves (Poumarat F et al (1999) Proceedings of International Symposium of Ruminant Mycoplasmas, Toulouse, June 1999 p65).

GB 1 074 920 is concerned with a vaccine for a pleuropneumonia-like organism (PPLO), which is suitable for human use, and a growth media for Mycoplasma pneumoniae. In this case the Mycoplasma pneumoniae is incubated in a sterile medium including evaporated chloroform extract of chicken egg yolk until antibody-inducing activity is imparted thereto, inactivated by use of formalin and concentrated. However, such formalised vaccines are not generally seen as effective. In addition, the vaccine described is for a specific human Mycoplasma and would not be sufficiently protective against bovine pneumonias in animals.

A number of attempts have been made to formulate a vaccine for Mycoplasmas, which are the causative agent for bovine pneumonia. None has, however, been particularly successful.

The applicants have surprisingly found, however, that by inactivating a strain of Mycoplasma, which is a causative agent of bovine pneumonias with saponin, particularly effective vaccines may be produced.

According to the present invention, there is provided a strain of Mycoplasma, which is a causative agent of bovine pneumonias, which has been inactivated by treatment with an agent, which may disrupt cell membranes, in particular saponin.

The cell membrane is preferably only partially disrupted and is not totally disrupted. In this way, the cells are prevented from growing yet retain the key antigenic regions of the organisms, which stimulate an immune response in the animal.

Unlike the production of some other vaccines, which may include the step of lyophilisation, it is not necessary to lyophilise the Mycoplasma in order to produce the vaccine according to the present invention.

As used herein, the term “saponin” refers to sapogenin glycosides, which may be derived from plants such as Quillaga bark. A range of saponins containing various amounts of sapogenin are available from Sigma.

These compounds are known to have hemolytic activity and may be used as adjuvants for vaccines.

Other cell membrane disrupters, such as those known in art may also be used.

Preferably, the strain of Mycoplasma, which is a causative agent of bovine pneumonias is a strain of Mycoplasma bovis or a Mycoplasma mycoides subsp. mycoides SC. In this regard, an inactivated strain of M. bovis is used in vaccines to protect against pneumonias caused by M. bovis and an inactivated strain of M. mycoides subsp. mycoides SC is used in vaccines to protect against contagious bovine pleuropnemonia which is caused by M. mycoides.

Strains of Mycoplasma bovis are available from a variety of sources. However, suitable strains may be isolated from cattle, in particular from the lungs of cattle who have suffered from pneumonia Mycoplasma bovis strain 96B/96 used in the present application was isolated from the lungs of a calf who had died of pneumonia

Strains of Mycoplasma mycoides subsp. mycoides SC are available from reference laboratories such as the Veterinary Laboratories Agency. Suitable strains may be isolated from the lungs of cattle which have been affected by African and not European outbreaks.

Strains of the invention may be used in vaccines.

An advantage of the present invention is that it is surprisingly a highly effective vaccine. In addition, as the vaccines of the present invention are inactivated, they are inherently safer and more stable than live vaccine products.

Thus, a further aspect of the invention comprises a veterinary composition comprising an inactivated strain of Mycoplasma which is a causative agent of bovine pneumonias as described above, in combination with a veterinarily acceptable carrier. Preferably, the strain is M. bovis or a M. mycoides subsp. mycoides SC.

The carrier may be solid or liquid, depending upon the intended mode of administration of the vaccine. Generally, however, the vaccine will be intended for parenteral administration. Liquid carriers, such as sterile aqueous or oily carriers for intravenous, subcutaneous, or intramuscular dosing may, therefore, be preferred.

Thus, the veterinary compositions of the present invention are suitably in the form of a sterile injectable aqueous or oily suspension, which may be formulated according to known procedures using one or more of the appropriate dispersing or wetting agents and suspending agents. A sterile injectable preparation may also be a sterile injectable solution or suspension in a non-toxic parenterally-acceptable diluent, for example a suspension in 1,3-butanediol.

Examples of suspending agents which may be used in the formulation of the invention include sodium carboxymethylcellulose, methylcellulose, hydroxypropylmethylcellulose, sodium alginate, polyvinyl-pyrrolidone, gum tragacanth and gum acacia. The aqueous suspensions may also contain one or more preservatives (such as ethyl or propyl p-hydroxybenzoate or anti-oxidants (such as ascorbic acid).

Preferably, the veterinary composition comprises only a strain of Mycoplasma according to the present invention and saponin in a buffered saline solution.

Oily suspensions may be formulated by suspending the active ingredient in a vegetable oil (such as arachis oil, olive oil, sesame oil or coconut oil) or in a mineral oil (such as liquid paraffin).

Dispersible powders and granules suitable for preparation of an aqueous suspension by the addition of water generally contain the active ingredient together with a dispersing or wetting agent, suspending agent and one or more preservatives.

The amount of active ingredient that is combined with one or more excipients to produce a single dosage form will necessarily vary depending upon the host treated and the particular route of administration. For example, a liquid formulation intended for subcutaneous administration to cattle and particularly calves, will generally contain, for example, from 0.1 mg to 0.5 mg/ml of a strain of inactivated Mycoplasma which is a causative agent of bovine pneumonias, compounded with an appropriate and convenient amount of excipients. Dosage unit forms will generally contain about 0.1 mg to about 2 mg, suitably from about 0.2 mg to 1 mg of a strain of inactivated Mycoplasma, which is a causative agent of bovine pneumonias strain. The strains are preferably the M. bovis or M. mycoides subsp. mycoides SC In this regard, an inactivated strain of M. bovis is used in vaccines to protect against calf pneumonia found in Europe and an inactivated strain of M. mycoides subsp. mycoides SC is used in vaccines to protect against contagious bovine pleuropnemonia found in Africa.

The size of the dose for prophylactic purposes will naturally vary according to the nature, the age of the animal and the route of administration, according to well known principles of medicine. Generally speaking however, the composition will be administered to young calves.

The composition of the invention may comprise further components, which would be known in the art. They may, for example include adjuvants such as aluminium compounds or Freund's incomplete adjuvant. Saponin, when present in the composition as a result of its use as an inactivating agent, will, however, also have an adjuvant effect, and it is unlikely, therefore, that further adjuvants would be required.

Where present, the saponin will be suitably be present in a liquid composition of the invention in an amount of at least 1 mg/ml in order to produce an adjuvant effect Preferably it will be present in an amount of from about 1-10 mg/ml, and more suitably at about 2 mg/ml.

In a further aspect, the invention provides a method of preparing an inactivated strain of Mycoplasma, which is a causative agent of bovine pneumonias as described above, which method comprises incubating a strain of Mycoplasma, which is a causative agent of bovine pneumonias in the presence of an agent which disrupts cell membranes such as saponin. Preferably, the strain is M. bovis or M. mycoides subsp. mycoides SC.

The amount of cell membrane disrupting agent present during the incubation should be sufficient to inactivate the strain and this will therefore depend to a large extent on the concentration of the strain within the culture, the precise nature of the strain, the particular agent used etc. However, where saponin is the agent, it should in general be present in the incubation culture in an amount of from about 0.5-10 mg/ml, preferably at about 2 mg/ml.

Once treated in this way, the inactivated strain may be isolated and formulated as discussed above. However, the direct product may be used as a vaccine formulation, provided all the components are veterinarily acceptable.

Where this is the case, saponin is present in the formulation and may exert an adjuvant effect.

In yet a further aspect, the invention provides a method of preventing disease in cattle caused by a strain of Mycoplasma, which is a causative agent of bovine pneumonias, said method comprising administering to the cattle a veterinary composition as described above. Preferably, the strain is M. bovis or M. mycoides subsp. mycoides SC.

Suitably, the composition will be administered as a single dose, but booster doses may be administered if required or necessary, and these may be determined by usual clinical methods. For parenteral administration, dosage units as described above may be used.

The invention will now be particularly described by way of example.

### EXAMPLE 1

Preparation of Mycoplasma bovis Vaccine

Eaton's broth medium was prepared by dissolving Difco PPLO broth base (21 g) (without crystal violet) in 700 mls of distilled water. Freshly prepared yeast extract (100 mls) was added together with unheated horse serum (200 mls), glucose (10 g), and penicilin (0.5 mls of 200,000 IU/ml), 0.2% phenol red (12.5 mls) and 0.02% of DNA. The pH was adjusted to 7.6-7.8 and sterilised by fitration

A universal container with a frozen aliquot of the master seed preparation (10^{th} passage of Mycoplasma bovis strain 86B/96 isolated from the lungs of a calf which had died of pneumonia), kept in 10 ml volumes at −70° C., was thawed completely in warm water.

This was added to 90 mls of Eaton's broth medium, prepared as described above, and incubated for 48 hours at 38.5° C. This was subcultured into a 900 ml volume of Eaton's medium and incubated at 38.5° C. for 48 hours. Cells were centrifuged at 10,000 g for 30 minutes and resuspended in the same volume of phosphate buffered saline (PBS pH 7.2). The cells were centrifuged as before, resuspended in 1/10th of the original volume in PBS and adjusted to contain approximately 0.5 mg/ml (approximately 10⁸ colony forming units per ml).

The mycoplasma cells were inactivated with saponin at 2 mg/ml overnight at 37° C. The suspension was aliquoted into 20 ml volumes and stored at 4° C.

### EXAMPLE 2

Biological Testing

The vaccine (2 ml), prepared as described in Example 1, was inoculated subcutaneously into each calf of four groups of seven or eight calves at about 4 weeks of age.

The four groups of cattle were housed separately.

Group A (vaccinated/challenged) containing 7 calves was inoculated with 2 mls of vaccine subcutaneously and challenged 3 weeks later with aerosol administration of virulent M. bovis (strain 5063) on consecutive days

Group B (unvaccinated/challenged) containing 7 calves were injected with placebo and then challenged as for Group A

Group C (unvaccinated/not challenged controls) containing 8 calves were kept as uninoculated controls

Group D (vaccinated/not challenged) containing 7 calves were immunised as for Group A but not challenged. These were monitored for adverse effects and antibody response

The vaccine was shown to be safe for calves and did not cause adverse clinical effects in groups A and D. It produced a strong serological responses in groups A and D within 2-3 weeks of administration. All calves in group B developed clinical signs of pneumonia while group A showed fewer signs. There was a significant decrease in body weight gain in group B compared to group A and a significant increase in lung lesions and rectal temperatures in group B calves.

In conclusion the M. bovis vaccine produced a significant level of protection against a virulent challenge.

### EXAMPLE 3

Preparation of Mycoplasma mycoides subsp. mycoides SC Vaccine

The Mycoplasma mycoides subsp. mycoides SC vaccine was prepared in the same way described in Example 1 for Mycoplasma bovis.

### EXAMPLE 4

Biological Testing

Cattle are vaccinated and then challenged by contact infection with infected cattle after a number of weeks.

All references mentioned in the above specification are herein incorporated by reference. Other modifications of the present invention will be apparent to those skilled in the art without departing from the scope and spirit of the invention. Although the invention has been described in connection with the specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

1. A strain of Mycoplasma which is a causative agent of bovine pneumonias which has been inactivated by treatment with an agent which disrupts cell membranes.

2. A stain according to claim 1 wherein the strain is Mycoplasma bovis.

3. A strain according to claim 1 wherein the strain is Mycoplasma mycoides subsp. mycoides SC.

4. A strain according to any one of claims 1 to 3 wherein the agent which disrupts cell membranes is saponin.

5. A veterinary composition comprising an inactivated strain of Mycoplasma which is a causative agent of bovine pneumonias as claimed in any one of claims 1 to 4, in combination with a veterinarily acceptable carrier.

6. A composition according to claim 5 wherein the strain is Mycoplasma bovis.

7. A composition according to claim 5 wherein the strain is Mycoplasma mycoides subsp. mycoides SC.

8. A composition according to any one of claims 5 to 7, which is suitable for parenteral administration.

9. A composition according to any one of claim 5 to 8 wherein the veterinary acceptable carrier is a liquid carrier.

10. A composition according to any one of claims 5 to 9 which further comprises one or more of a dispersing agent, a wetting agent or a suspending agent.

11. A composition according to any one of claims 5 to 10, which further comprises an adjuvant.

12. A composition according to claim 11 wherein the adjuvant is saponin.

13. A method of preparing an inactivated strain of Mycoplasma, which is a causative agent of bovine pneumonias as described above, which method comprises incubating a strain of Mycoplasma, which is a causative agent of bovine pneumonia in the presence of an agent which disrupts cell membranes.

14. A method according to claim 13 wherein the strain is Mycoplasma bovis.

15. A method according to claim 13 wherein the strain is Mycoplasma mycoides subsp. mycoides SC.

16. A method according to any one of claims 13 to 15 wherein the agent which disrupts cell membranes is saponin.

17. A method according to claim 16 wherein the saponin is present in the incubation culture in an amount of from about 0.5-10 mg/ml.

18. A method according to claim 17 wherein the saponin is present in an amount of about 2 mg/ml.

19. A method of preventing disease in cattle caused by a strain of Mycoplasma, which is a causative agent of bovine pneumonias, said method comprising administering to the cattle an inactivated strain of Mycoplasma, which is a causative agent of bovine pneumonias according to any one of claims 1 to 4 or a veterinary composition as according to any one of claims 5 to 12.

20. The use of a strain of Mycoplasma, which is a causative agent of bovine pneumonias according to any one of claims 1 to 4 in the preparation of a medicament for the treatment of animals.

21. A strain of Mycoplasma, which is a causative agent of bovine pneumonias substantially as hereinbefore described with reference to the examples.
